(19) United States
(12) Patent Application Publication
(10) Pub. No.: US 2026/0275528 A1
(43) Pub. Date: Sep. 17, 2026

(54) ROOM-TEMPERATURE-OPERATED HIGH-SELECTIVITY NO2 SENSOR DEVICE AND FABRICATION METHOD THEREOF

(71) Applicant: SHENZHEN TECHNOLOGY UNIVERSITY, Shenzhen (CN)

(72) Inventors: Fang XU, Shenzhen (CN); Ke JIANG, Shenzhen (CN); Xuelan CHENG, Shenzhen (CN); Yizheng LIU, Shenzhen (CN)

(73) Assignee: SHENZHEN TECHNOLOGY UNIVERSITY, Shenzhen (CN)

(21) Appl. No.: 19/674,183

(22) Filed: May 12, 2026

(30) Foreign Application Priority Data
Mar. 13, 2025 (CN) 202510294484.3

(51) Int. Cl. C23C 26/00; G01N 33/00

(52) U.S. Cl. CPC C23C 26/00; G01N 33/0027

(57) ABSTRACT

A room-temperature-operated high-selectivity NO₂ sensor device and a fabrication method thereof are provided. The room-temperature-operated high-selectivity NO₂ sensor device includes an electrode substrate and a black TiO₂ sensing film uniformly loaded on a surface of the electrode substrate. The fabrication method includes the following steps: adding TiO₂ to a solvent to prepare a slurry; drop-coating the slurry uniformly onto an electrode substrate, and uniformly and slowly heating the electrode substrate to remove the solvent; repeating the drop-coating and heating process a plurality of times to form a TiO₂ coating with a desired thickness on a surface of the electrode substrate; and vacuum-annealing the electrode substrate with the TiO₂ coating to form a black TiO₂ sensing film on the surface of the electrode substrate. The black TiO₂ sensing film has oxygen vacancies, enabling NO₂ detection at room temperature with high selectivity and humidity resistance.

### CROSS REFERENCE TO THE RELATED APPLICATIONS

This application is based upon and claims priority to Chinese Patent Application No. 202510294484.3, filed on Mar. 13, 2025, the entire contents of which are incorporated herein by reference.

### TECHNICAL FIELD

The present disclosure relates to the technical field of gas sensors, and specifically relates to a room-temperature-operated high-selectivity NO₂ sensor device, and a fabrication method thereof.

### BACKGROUND

Nitrogen dioxide (NO₂), a common hazardous gas, primarily originates from fossil fuel combustion, vehicle exhaust emissions, and industrial production processes. Nitrogen dioxide in the atmosphere not only contributes to the generation of acid rain but also undergoes photochemical reactions with volatile organic compounds (VOCs) under sunlight to produce ozone and fine particulate matter (PM_{2·5}), thereby exacerbating air pollution and compromising air quality. More seriously, NO₂ poses significant hazards to human health. The long-term exposure to high-concentration NO₂ can lead to respiratory diseases, cardiovascular diseases, and even an increased risk of cancer. NO₂ can also adversely affect plant growth, inhibit the yield and quality of crops, and cause damage to ecosystems. Monitoring demands and technical challenges: As the public concern for environmental protection and health continues to grow, the real-time and accurate monitoring of a NO₂ concentration in the air has become particularly important. Traditional NO₂ detection methods include, for example, chemiluminescence and spectrophotometry, which show high sensitivity and high accuracy. However, these methods have disadvantages including complex equipment, cumbersome operations, high costs, and difficulty in achieving rapid on-site detection. Therefore, developing a sensor capable of efficiently, sensitively, and selectively detecting NO₂ at room temperature is of great significance for fields such as environmental monitoring, industrial safety, and health protection.

Traditional metal oxide semiconductor gas sensors are characterized by rapid response and easy integration, but typically require high-temperature operation, leading to increased energy consumption and aggravated device complexity. In addition, existing sensors all suffer from issues such as a lack of gas selectivity and humidity resistance, making these sensors fail to effectively identify NO₂ in mixed gases or high-humidity environments. In some studies, the room-temperature NO₂ response is acquired and the NO₂ selectivity is improved to some extent through composite preparation or doping. However, these studies still cannot achieve the identification of NO₂ and offer no humidity resistance. Moreover, the preparation of a composite increases the production cost of a sensor. Precisely based on this need, the present disclosure proposes a novel room-temperature-operated NO₂ detection technology, which aims to overcome the shortcomings of the prior art through innovative material design and achieve the detection of a NO₂ with high sensitivity, high selectivity, and a low detection limit. The present disclosure also provides a method for enhancing response sensitivity, selectivity, and humidity resistance of NO₂ detection.

### SUMMARY

An objective of the present disclosure is to provide a room-temperature-operated high-selectivity NO₂ sensor device, a fabrication method thereof, and a method for improving response sensitivity, selectivity, and humidity resistance of NO₂ detection. The technical problem to be solved by the present disclosure is the low selectivity of existing metal oxides-based gas-sensitive elements for NO₂.

To achieve the above objective, the present disclosure adopts the following technical solutions:

A room-temperature-operated high-selectivity NO₂ sensor device is provided, including an electrode substrate and a black TiO₂ sensing film uniformly loaded on a surface of the electrode substrate, where the black TiO₂ sensing film is prepared by vacuum-annealing a TiO₂ coating.

A fabrication method of a room-temperature-operated high-selectivity NO₂ sensor device is provided, including the following steps:
(1) adding TiO₂ to a solvent to prepare a slurry; drop-coating the slurry uniformly onto an electrode substrate, and slowly heating the electrode substrate on a hot plate to remove the solvent; and repeating the drop-coating and heating process a plurality of times to form a TiO₂ coating with a desired thickness on a surface of the electrode substrate; and
(2) vacuum-annealing an electrode substrate with the TiO₂ coating to form a black TiO₂ sensing film on the surface of the electrode substrate.

Preferably, in the step (2), the vacuum-annealing is conducted at 450° C. to 550° C. for 2 h to 6 h; and more preferably, the vacuum-annealing is conducted at 500° C. for 4 h.

Preferably, in the step (1), the TiO₂ is nano-TiO₂ with a burr-like structure on a surface, which is prepared by the following process: adding (NH₄)₂TiF₆ and H₃BO₃ to water to prepare a mixed solution; immersing ZnO in the mixed solution at room temperature until a white product is generated at a bottom of the solution; and conducting centrifugation and washing to produce the nano-TiO₂ with the burr-like surface, where a molar ratio of the (NH₄)₂TiF₆, the H₃BO₃, and the ZnO is 1:(2.8-3.3):(0.2-0.25).

In the step (1), the solvent includes, but is not limited to, deionized (DI) water and ethanol.

In the step (1), the electrode substrate is heated on the hot plate at a temperature 20° C. to 40° C. lower than a boiling point of the solvent. This temperature allows for slow heating of the electrode substrate, thereby preventing the sensing film from defects such as cracks due to excessive or too-rapid heating.

Preferably, in the step (1), the drop-coating and heating process is repeated 2 times to 8 times.

Compared with the prior art, the present disclosure has the following beneficial effects: (1) The fabrication method is simple, and requires only vacuum annealing to produce a black TiO₂ sensing film. (2) The black TiO₂ sensing film has abundant oxygen vacancies, enabling NO₂ detection at room temperature with high sensitivity and a low detection limit down to the sub-ppb level. (3) The room-temperature-operated high-selectivity NO₂ sensor device exhibits excellent selectivity for NO2, and can effectively distinguish NO₂ from other common gases. (4) The room-temperature-operated high-selectivity NO₂ sensor device can achieve rapid and efficient NO2 detection with low cost, and is suitable for widespread application. (5) The room-temperature-operated high-selectivity NO₂ sensor device exhibits excellent humidity resistance to NO₂.

### BRIEF DESCRIPTION OF THE DRAWINGS

FIG. 1 shows X-ray diffraction (XRD) patterns of W-TiO₂ and B-TiO₂;

FIGS. 2A-2B show scanning electron microscopy (SEM) images of W-TiO₂ and B-TiO₂;

FIG. 3 shows electron paramagnetic resonance (EPR) spectra of W-TiO₂ and B-TiO₂; and

FIGS. 4A-4B show comparison of selectivity of W-TiO₂ and B-TiO₂ for a NO₂ gas and other gases,

FIGS. 5A-5B show responses of W-TiO₂ to 0.5 ppm NO₂ under different humidity conditions in FIG. 5A, and responses of B-TiO₂ to 10 ppm NO₂ under different humidity conditions in FIG. 5B.

### DETAILED DESCRIPTION OF THE EMBODIMENTS

The present disclosure is further described below with reference to the accompanying drawings and specific examples.

### Example 1

A fabrication method of a room-temperature-operated high-selectivity NO₂ sensor device was provided, specifically including the following steps:
(1) (NH₄)₂TiF₆ and H₃BO₃ were added to water to prepare a mixed solution. Then, ZnO was immersed in the mixed solution at room temperature for 20 h to 50 h until a white product was generated at a bottom of the solution. A molar ratio of (NH₄)₂TiF₆, H₃BO₃, and ZnO was 1:3:0.2. The white product was separated from a reaction solution by centrifugation and rinsed three times with DI water to finally produce nano-TiO₂ with a burr-like structure on a surface.
(2) TiO₂ was mixed with DI water to prepare a slurry. 3 μL of the slurry was drop-coated uniformly onto an electrode substrate (such as an alumina ceramic substrate). After the drop-coating, the electrode substrate was heated on a hot plate at 70° C. for 5 min to remove the water. This drop-coating and heating process was repeated three times to form a TiO₂ coating on a surface of the electrode substrate.
(3) An electrode substrate with the TiO₂ coating was annealed in air at 500° C. for 4 h to produce white TiO₂, which was denoted as W-TiO₂.

### Example 2

This example was the same as Example 1 except for the step (3):
(3) An electrode substrate with the TiO₂ coating was vacuum-annealed at 500° C. for 4 h to produce black TiO₂, which was denoted as B-TiO₂.

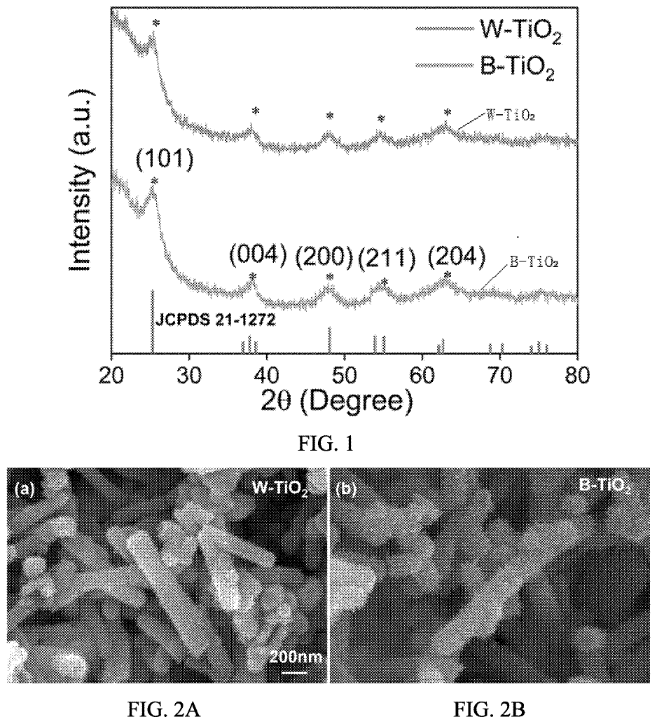

XRD patterns of W-TiO₂ and B-TiO₂ are shown in FIG. 1. All diffraction peaks in the XRD patterns of the two samples can be clearly indexed to an anatase structure of TiO₂ (JCPDS card No. 21-1272), and no additional peaks or phases are observed. B-TiO₂ has more pronounced diffraction peaks than W-TiO₂, indicating excellent crystallinity of B-TiO₂.

FIGS. 2A-2B show SEM images of W-TiO₂ and B-TiO₂. There is virtually no difference in SEM morphology between products obtained after annealing in different environments. Both W-TiO₂ and B-TiO₂ retain the burr-like structure of TiO₂, which increases the specific surface area of the room-temperature-operated high-selectivity NO₂ sensor device.

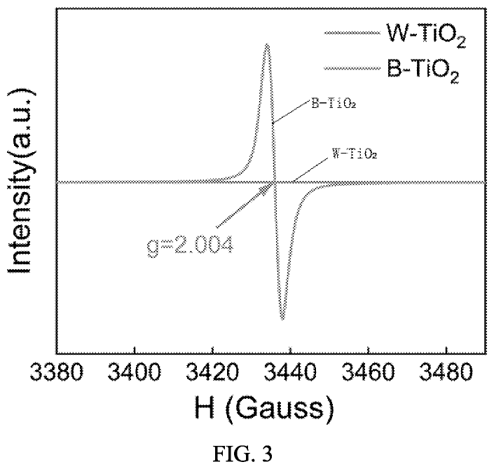

W-TiO₂ and B-TiO₂ were each subjected to EPR spectroscopy. As shown in FIG. 3, B-TiO₂ presents a strong EPR signal at g=2.004, which is attributed to the trapping of electrons in oxygen vacancies. However, such a signal is barely observable in W-TiO₂, confirming that B-TiO₂ has more oxygen vacancies than W-TiO₂.

The sensing performance of sensors based on W-TiO₂ and B-TiO₂ towards NO₂ and several common interfering gases at room temperature was tested. The electrode substrates loaded with W-TiO₂ or B-TiO₂ fabricated above were each placed in a sealed chamber filled with test gases, and a response of a sensor was measured. The test gases were ethanol, isopropanol, acetone, carbon dioxide (CO₂), hydrogen (H₂), formaldehyde, and NO₂. A response of a sensor is defined as follows: When Rₐ>R_{g}, response=(Rₐ−R_{g})/R_{g}×100%. When Rₐ<R_{g}, response=(R_{g}−Rₐ)/Rₐ×100% (where Rₐ and R_{g} represent output resistance of the sensor before and after gas exposure, respectively).

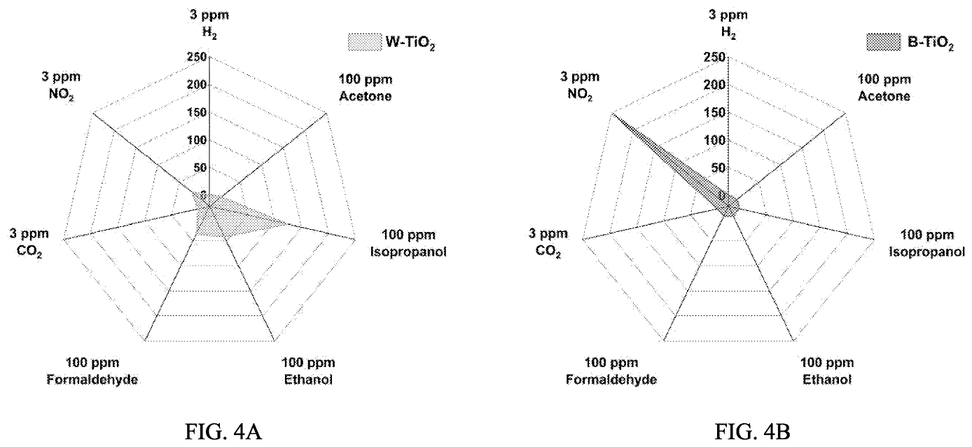

FIGS. 4A-4B are radar charts of the comparison of selectivity of W-TiO₂ (FIG. 4A) and B-TiO₂ (FIG. 4B) for NO₂ and other gases, summarizing responses of W-TiO₂ and B-TiO₂ to ethanol, isopropanol, acetone, carbon dioxide (CO₂), hydrogen (H₂), formaldehyde, and NO₂. It can be seen that responses of W-TiO₂ and B-TiO₂ to 3 ppm NO₂ are 20.9% and 250%, respectively. A response of B-TiO₂ is 12 times a response of W-TiO₂. In addition to the improved response, the selectivity of B-TiO₂ toward NO₂ is significantly enhanced. B-TiO₂ suppresses responses to the ethanol, isopropanol, acetone, carbon dioxide (CO₂), hydrogen (H₂), and formaldehyde, resulting in almost no response to the ethanol, isopropanol, acetone, carbon dioxide (CO₂), hydrogen (H₂), and formaldehyde. Compared with W-TiO₂, the B-TiO₂ sensor exhibits ultra-high selectivity towards NO₂, and enables the identification of NO₂ in a mixed gas of seven gases.

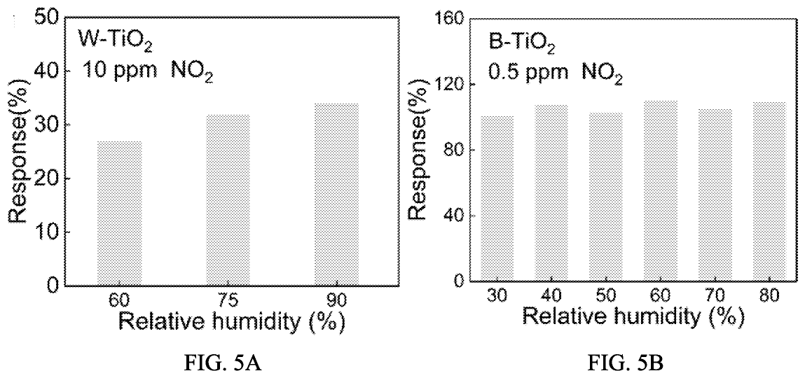

According to the sensing performance of W-TiO₂ and B-TiO₂ towards NO₂ under different humidity conditions (FIG. 5A and FIG. 5B), the response of W-TiO₂ to NO₂ increases with the increase in humidity (FIG. 5A). In contrast, the response of B-TiO₂ to NO₂ within a relative humidity range of 30% to 80% is not affected by atmospheric moisture (FIG. 5B). Compared with W-TiO₂, B-TiO₂ exhibits significant humidity resistance in NO₂ detection.

What is claimed is:

1. A fabrication method of a room-temperature-operated high-selectivity NO₂ sensor device, comprising the following steps:
(1) adding TiO₂ to a solvent to prepare a slurry; drop-coating the slurry uniformly onto an electrode substrate, and uniformly and slowly heating the electrode substrate to remove the solvent; and repeating a drop-coating and heating process a plurality of times to form a TiO₂ coating with a desired thickness on a surface of the electrode substrate, wherein in the step (1), the TiO₂ is nano-TiO₂ with a burr-like structure on a surface; and
(2) vacuum-annealing an electrode substrate with the TiO₂ coating to form a black TiO₂ sensing film on the surface of the electrode substrate, wherein in the step (2), the annealing is conducted at 450° C. to 550° C.

2. The fabrication method of the room-temperature-operated high-selectivity NO₂ sensor device according to claim 1, wherein the annealing is conducted for 2 h to 6 h.

3. The fabrication method of the room-temperature-operated high-selectivity NO₂ sensor device according to claim 1, wherein the annealing is conducted at 500° C. for 4 h.

4. The fabrication method of the room-temperature-operated high-selectivity NO₂ sensor device according to claim 1, wherein a process for preparing the nano-TiO₂ with the burr-like structure is as follows: adding (NH₄)₂TiF₆ and H₃BO₃ to water to prepare a mixed solution; immersing ZnO in the mixed solution at room temperature until a white product is generated at a bottom of the solution; and conducting centrifugation and washing to produce the nano-TiO₂ with the burr-like structure on the surface, wherein a molar ratio of the (NH₄)₂TiF₆, the H₃BO₃, and the ZnO is 1:(2.8-3.3):(0.2-0.25).

5. The fabrication method of the room-temperature-operated high-selectivity NO₂ sensor device according to claim 1, wherein in the step (1), the solvent is deionized (DI) water or ethanol.

6. The fabrication method of the room-temperature-operated high-selectivity NO₂ sensor device according to claim 1, wherein in the step (1), the electrode substrate is heated on a hot plate at a temperature 20° C. to 40° C. lower than a boiling point of the solvent.

7. The fabrication method of the room-temperature-operated high-selectivity NO₂ sensor device according to claim 1, wherein an oxygen vacancy content of the TiO₂ is increased by controlling annealing conditions.

8. A room-temperature-operated high-selectivity NO₂ sensor device fabricated by the fabrication method according to claim 1, comprising the electrode substrate and the black TiO₂ sensing film uniformly loaded on the surface of the electrode substrate, wherein the black TiO₂ sensing film is prepared by vacuum-annealing the TiO₂ coating.

9. The room-temperature-operated high-selectivity NO₂ sensor device according to claim 8, wherein in the fabrication method, the annealing is conducted for 2 h to 6 h.

10. The room-temperature-operated high-selectivity NO₂ sensor device according to claim 8, wherein in the fabrication method, the annealing is conducted at 500° C. for 4 h.

11. The room-temperature-operated high-selectivity NO₂ sensor device according to claim 8, wherein in the fabrication method, a process for preparing the nano-TiO₂ with the burr-like structure is as follows: adding (NH₄)₂TiF₆ and H₃BO₃ to water to prepare a mixed solution; immersing ZnO in the mixed solution at room temperature until a white product is generated at a bottom of the solution; and conducting centrifugation and washing to produce the nano-TiO₂ with the burr-like structure on the surface, wherein a molar ratio of the (NH₄)₂TiF₆, the H₃BO₃, and the ZnO is 1:(2.8-3.3):(0.2-0.25).

12. The room-temperature-operated high-selectivity NO₂ sensor device according to claim 8, wherein in the fabrication method, in the step (1), the solvent is DI water or ethanol.

13. The room-temperature-operated high-selectivity NO₂ sensor device according to claim 8, wherein in the fabrication method, in the step (1), the electrode substrate is heated on a hot plate at a temperature 20° C. to 40° C. lower than a boiling point of the solvent.

14. The room-temperature-operated high-selectivity NO₂ sensor device according to claim 8, wherein in the fabrication method, an oxygen vacancy content of the TiO₂ is increased by controlling annealing conditions.
